(19) United States
(12) Patent Application Publication
(10) Pub. No.: US 2020/0038672 A1
(43) Pub. Date: Feb. 6, 2020

(54) RADIOFREQUENCY BALLOON CATHETER SYSTEM

(71) Applicant: JAPAN ELECTEL INC., Nishi-ku, Yokohama-shi, Kanagawa (JP)

(72) Inventor: Shutaro SATAKE, Kamakura-shi Kanagawa (JP)

(21) Appl. No.: 15/739,850

(22) PCT Filed: Dec. 16, 2015
(86) PCT No.: PCT/JP2015/085239

§ 371 Date: Dec. 26, 2017

(51) Int. Cl. A61N 1/40; A61M 25/10

(52) U.S. Cl. CPC A61N 1/403; A61M 25/104; A61M 2205/3368; A61M 2205/366; A61M 2205/0216; A61M 2205/0238; A61M 2205/3317; A61M 2205/3606

(57) ABSTRACT

Provided is a radiofrequency heating balloon catheter system including a balloon (6) arranged between distal ends of an inner tube (2) and an outer tube (3), an electrode for delivery of radiofrequency current (11) arranged inside the balloon (6), and a temperature sensor (12) arranged at a distal portion of the inner tube inside the balloon in contact with a film of the balloon for estimating the surface temperature of the balloon. Also, the inner tube inside the balloon has a plurality of nozzles (7) bored through in a region extending from the distal portion of the inner tube to the proximal portions thereof to forcibly cool the inside of the balloon and the electrode for delivery of radiofrequency current. This system can be utilized for the application of liminal organs to maintain the surface temperature of the balloon at 45° C. or lower to thereby heat a luminal organ media at a temperature of 45 to 50° C. without damaging endothelial cells, in contact with the balloon film, in order to soften the media collagen by the balloon inflated under a relatively low pressure of 3 to 6 atmospheres with minimal cellular damages to thereby dilate the luminal without causing any vascular dissection. The present radiofrequency heating balloon catheter system performs luminal formation under relatively low pressure and temperature using radiofrequency heating and in-balloon perfusion. As the result, no acute occlusion occurs due to a dissection of luminal walls, and damages to the intima and media cells are minimized. Further, restenosis originating from an infiltration of inflammatory cells is restrained.

### TECHNICAL FIELD

The present invention relates to a radiofrequency balloon catheter system for thermally dilating a stenosis site with a deflated balloon being interposed into the stenosis site within a hollow organ by irradiating the stenosis site with a radiofrequency electric field emanating from an in-balloon electrode and applying a pressure to the balloon while protecting an intima with a coolant perfusing through the balloon.

### BACKGROUND

Many of stenoses, such as coronary artery stenosis that cause angina or myocardial infarction, are known to be due to arteriosclerotic lesions in a vascular membrane. Stenosis site dilatation with conventional balloon causes a dissection in tissues. For this reason, stent insertion is performed to avoid acute occlusion originating from vascular dissociation or recoil. In this regard, if they are dilated while applying a heat thereto using a radiofrequency hot balloon catheter, such stenoses are improved without causing any vascular dissociation or recoil, thereby eliminating a need for such stent. One example of ablation systems employing such radiofrequency hot balloon catheter is disclosed in e.g., Patent document 1.

In conventional radiofrequency hot balloon catheters, stenosis site dilatation is performed in such a way that the balloon is deflated and inserted into a vascular stenosis site before inflating and pressurizing the balloon to dilate the vascular stenosis, while heating the site by applying thereto a radiofrequency energy from an electrode inside the balloon to fuse collagen tissues and atheroma, etc. therein. Whilst such method as to dilate a vessel at a relatively low pressure while heating the vessel to soften and fuse a lesion therein has an advantage that the method does not cause vascular dissociation or recoil, and hence it is free from a risk of developing acute occlusion. The method, however, has a problem that restenosis may occur due to cell proliferation associated with an inflammatory response caused by cell ablation.

In order to prevent damages to intima constituting blood vessel, there have been developed balloon cooling methods using perfusion inside a balloon. Such methods include a method of perfusing a balloon interior through an outer tube and an inner tube of a catheter shaft, as disclosed in Patent Document 2, and a method of performing perfusion between the inside and the outside of a balloon through pores in a balloon film, as disclosed in patent document 3, both of which were invented by the inventor of the present invention.

### PRIOR ART DOCUMENT

### Patent Document

### Patent Document 1: Japanese Patent Application Publication No. 2002-126096

Patent Document 2: U.S. Pat. No. 6,952,615
Patent Document 3: U.S. Pat. No. 6,491,710

### SUMMARY OF THE INVENTION

### Problem to be Solved by the Invention

It has been found that in-balloon cooling while heating the same using a radiofrequency balloon catheter may prevent damages to an intima of a luminal organ, but intense ablation onto media cells also induces an inflammatory reaction, thus triggering vascular restenosis. To date, such restenosis, originated from the proliferation thereof, has been thought to be avoided if only media smooth muscle is ablated. However, it has also been found that the inflammatory cells are also mobilized from bone marrow-derived cells and accumulated in the luminal organ to thereby cause restenosis.

The present invention has been achieved in light of the above problem. Various experiments on animals were conducted for minimizing damages to the cell constituting luminal organs while softening and pressure-stretching collagen that is an adhesive material of luminal organs. As a result of them, it has been verified in a tank experiment that forcible perfusion inside a balloon while delivering radiofrequency current from outside to the electrode in the balloon, having a length of 20 mm and a diameter of 5 mm, allows a balloon to maintain surface temperature of 45° C. or lower, and that the radiofrequency electric field, irradiated onto the balloon surroundings, heats surrounding tissues, located at 1 mm distance away from the balloon surface, at a temperature of 45 to 50° C. It has also been found that if a luminal organ wall maintains a temperature of 45 to 50° C. for the heating period of 30 to 60 seconds, then collagen—an adhesive material of luminal organs—is allowed to be softened by heat with minimal damages to wall-constituting cells in order to dilate a lumen of a luminal organ using inflation of a balloon under relatively low pressure of 3 to 6 atmospheres without causing any luminal organ dissection. It is an object of the present invention to provide a radiofrequency balloon catheter system for achieving such functionality.

### Solution to Problem

The present radiofrequency heating balloon catheter system includes an electrode for delivery of radiofrequency current inside a balloon provided in a distal of the catheter shaft, and a plurality of nozzles are perforated through an inner tube inside the balloon for discharging in-balloon solution to forcibly cool the electrode for delivery of radiofrequency current, arranged on the inner tube, by the perfusion flowing in and out of the inner tube. Further, a temperature sensor is arranged at a distal portion of the inner tube inside the balloon in contact with the balloon membrane to thereby allow one to know the surface temperature of the balloon in a near accurate manner. This system can be utilized for vascular application to maintain the surface temperature of the balloon at 45° C. or lower to thereby heat a luminal organ media at a temperature of 45 to 50° C. for 30 to 60 seconds without damaging endothelial cells, in contact with the balloon film, in order to soften and stretch the collagen under a relatively low pressure of 3 to 6 atmospheres with minimal cellular damages to thereby dilate the stenosis site without causing any vascular dissection. Even after the cessation of radiofrequency current delivery, pressures and perfusion cooling inside the balloon are continued to be provided to restrain the inflammatory response at the heat-treated site and to memorize the shape of collagen in the stretched state to thereby keep the luminal organ in a dilated state.

According to the system for cooling the inside of the balloon while heating the outside of the balloon, the system may employ a conventional balloon catheter members, as well as an electrode of fine wire in order to use the system for the treatment of vascular stenosis site such as those for coronary angioplasty without significantly changing the profile of the balloon.

A first aspect of the present invention is drawn to a radiofrequency balloon catheter system including:

a catheter shaft comprising an inner tube and an outer tube;

a balloon that is inflatable and deflatable and provided between distal ends of the inner tube and the outer tube;

an electrode for delivery of radiofrequency current provided within the balloon;

a plurality of in-balloon perfusion nozzles bored in the inner tube inside the balloon;

a temperature sensor arranged at a distal portion of the inner tube inside the balloon, the temperature sensor being in contact with a film of the balloon;

a radiofrequency generator and a thermometer respectively connected to the electrode for delivery of radiofrequency current and the temperature sensor via electric wires within the catheter shaft;

a solution transport path defined by the outer tube and the inner tube, the solution transport path being in communication with an inside of the balloon, and connected to a perfusion pump for feeding a coolant into the balloon; and

a guide wire that is coated with an elastic material and insertable into the inner tube (FIGS. 1 to 4).

A second aspect of the present invention is drawn to the first aspect, wherein the plurality of in-balloon perfusion nozzles are arranged at distal and proximal portions of the inner tube, the nozzles arranged at the distal portion formed greater in size than the nozzles at the proximal portion, and perfusing greater amount of perfusate than the nozzles at the proximal portion (FIGS. 5A to 5E).

A third aspect of the present invention is drawn to the first aspect, wherein the system is configured to perfuse a coolant inside the balloon such that an intima of a target tissue, in contact with the balloon, is maintained at a temperature of 45° C. or lower and to irradiate a radiofrequency electric field such that a media of a target luminal organ is heated to a temperature of 45 to 50° C. (FIG. 6).

A fourth aspect of the present invention is drawn to the first aspect, wherein the system is configured to maintain an intima of a target tissue, in contact with the balloon, at a temperature of 45° C. or lower and to set an energization time for 30 to 60 seconds for minimizing damages to the intima. (FIGS. 7 to 10).

A fifth aspect of the present invention is drawn to the first aspect, wherein the system is configured to heat the media of a target luminal organ at a temperature of 45 to 50° C. for 30 to 60 seconds to soften collagen fibers while minimizing damages to the media cells in order to dilate a stenosis site using the balloon inflated under a relatively low pressure of 3 to 6 atmospheres without causing any dissociations or dissections. (FIGS. 7 to 10).

A sixth aspect of the present invention is drawn to the first aspect, wherein the system is configured to intermittingly deliver a radiofrequency current to the electrode for delivery of radiofrequency current or to deliver the same with a variation in intensity.

A seventh aspect of the present invention is drawn to the first aspect, wherein the electrode is installed in front and back of the balloon to measure an impedance between them.

### Advantageous Effects of Invention

FIGS. 1 to 4 and 5A to 5E illustrate schematic diagrams according to the first aspect of the present invention. When the guide wire is inserted into the inner tube of the catheter shaft, and the solution within the balloon is suctioned through the solution transport path, the balloon becomes deflated. Meanwhile, when the balloon is injected with perfusate, the balloon becomes inflated to discharge the perfusate through the nozzles, bored in the inner tube, to the outside. Upon delivery of radiofrequency current, the electrode for delivery of radiofrequency current inside the balloon uniformly radiates radio frequency electric field to thereby heat the surrounding of the balloon. In the meantime, the temperature sensor, arranged at a distal portion of the inner tube inside the balloon in contact with a balloon film, allows one to monitor a value close to the surface temperature of the balloon film.

In the second aspect of the present invention, in accordance with the first aspect, the inner tube inside the balloon has a plurality of nozzles bored through in a region extending from the distal portion of the inner tube to the proximal portions thereof, where the nozzles arranged at the distal portion are formed greater in size than those at the proximal portion. Accordingly, even if the balloon is tightly interposed in a stenosis site, perfusate inside the balloon continues to flow through the proximal nozzles into the inner tube to thereby cool the electrode for delivery of radiofrequency current from within. If, on the other hand, the balloon is inflated from the stenosis site, perfusate inside the balloon mostly passes through a region within the balloon before being discharged through the distal nozzles to the outside of the inner tube, thus cooling the electrode for delivery of radiofrequency current, and the balloon from within (FIGS. 5A to 5E).

In the third aspect of the present invention, in accordance with the first aspect, when the balloon has a length of 20 mm and a diameter of 2.5 to 5 mm, the coolant inside the balloon is set to be perfused at the perfusion rate of 10 to 50 cc per minute with the radiofrequency output of 20 to 80 W. In such condition, the temperature of an intima of a target tissue, in contact with the balloon, is maintained at 45° C. or lower, while a radiofrequency electric field irradiated to the same heats the media of the target luminal organ to the temperature of 45 to 50° C. As the result, the media collagen is softened without having any major influence, such as coagulative necrosis, on the cell constituent (FIG. 6).

In the fourth aspect of the present invention, in accordance with the first aspect, the temperature of an intima of a target tissue in contact with the balloon is kept at 45° C. or lower to thereby minimize damages to intima (FIGS. 7 to 10).

In the fifth aspect of the present invention, in accordance with the first aspect, the system is configured to heat a media of luminal organ at a temperature of 45 to 50° C. for 30 to 60 seconds to soften collagen fibers while minimizing damages to media cells in order to allow a stenosis site to be dilated using the balloon inflated at relatively low pressure of 3 to 6 atmospheres without causing any dissection in a target tissue media or vascular recoil. Accordingly, no complication of acute vascular occlusion occurs, and restenosis can be minimized even after the dilation (FIGS. 7 to 10).

In the sixth aspect of the present invention, in accordance with the first aspect, the system is configured to intermittingly deliver a radiofrequency current to the electrode for delivery of radiofrequency current or to deliver the same with a variation in intensity. As the result, a tissue in contact with the balloon can be heated to a high deep temperature.

In the seventh aspect of the present invention, in accordance with the first aspect, the electrode is installed in front and back of the balloon via electric wires to which an impedance measurement device is connected, allowing one to monitor a change exerted on tissues by the radiofrequency heating.

### BRIEF DESCRIPTION OF DRAWINGS

FIG. 1 is an explanatory drawing illustrating a main framework of the present radiofrequency balloon catheter system including a balloon provided in the vicinity of distal ends of the inner and outer tubes of a catheter, an electrode for delivery of radiofrequency current provided within the balloon, a plurality of in-balloon perfusion nozzles bored in at a distal portion of the inner tube inside the balloon and a temperature sensor arranged at the distal portion thereof in contact with a film of the balloon. Here, as the radiofrequency generator performs delivery of radiofrequency current between a counter electrode placed on the body surface and the electrode for delivery of radiofrequency current inside the balloon, a radio frequency electric field is radiated from the electrode for delivery of radiofrequency current to the surrounding. Also, as the in-balloon solution is injected by the liquid transfusing pump through a solution transport path, defined between the inner tube and the outer tube, the in-balloon solution is discharged through the distal nozzles of the inner tube to the outside, thereby cooling the balloon.

FIG. 2 is an explanatory drawing illustrating the balloon being inserted into a stenosis site as the balloon is filled with a perfusate inside.

FIG. 3 is an explanatory drawing illustrating how the electrode for delivery of radiofrequency current and inside of the balloon is cooled, as the balloon is infused with perfusate while delivering radiofrequency current to flow the perfusate through the proximal nozzles inside the balloon to the distal nozzle.

FIG. 4 is an explanatory drawing illustrating a blood vessel being dilated under a relatively low pressure of 3 to 6 atmospheres as the media collagen is thermally softened at 45 to 50° C. without damaging an intima of a blood vessel.

FIG. 5A is an explanatory drawing illustrating a radiofrequency balloon catheter system with nozzles that are bored in at distal and proximal portions of the inner tube inside the balloon.

FIG. 5B is an explanatory drawing illustrating a guide wire inside the inner tube of the balloon catheter being interposed in a vascular stenosis site.

FIG. 5C is an explanatory drawing illustrating the way in which an elastic guide wire is extended when suctioning the in-balloon solution to block the nozzles for deflating the balloon to thereby allow the wire to pass through the vascular stenosis site.

FIG. 5D is an explanatory drawing illustrating the way in which vascular media is primarily heated by the irradiation of the radiofrequency current onto the stenosis site to dilate the same. In the meantime, upon injection of in-balloon solution while delivering radiofrequency current, the in-balloon solution flows through the proximal nozzles into the inner tube and the distal portion of the balloon, and then is discharged through the distal end of the catheter, which allows perfusate to cool the electrode for delivery of radiofrequency current from within.

FIG. 5E is an explanatory drawing illustrating the way in which the injection rate is enhanced to further increase the internal pressure of the balloon to thereby thermally dilate a vascular stenosis site, allowing in-balloon solution to pass through the gap between the balloon and the stenosis site and to be discharged through the distal nozzles of the inner tube.

FIG. 6 shows a graph illustrating temperatures of a tissue, in contact with the balloon surface, simultaneously recorded at the depths of 1, 2 and 3 mm below the balloon surface while perfusing the inside of the balloon with perfusion of 30 cc per minute under a radiofrequency current of 40 W output, showing surface temperature of 45° C. or lower, and 1 mm deep temperature of 45 to 50° C.

FIG. 7 illustrates a perspective image of the balloon catheter interposed within a porcine iliac artery at a moment of being inflating and perfusing the inside of the balloon with the coolant, serving as a perfusate, at the rate of 30 cc per minute under 3 to 6 atmospheres while delivering radiofrequency current at an output of 40 W for one minute.

FIG. 8 illustrates an overall image of the tissue after angioplasty.

FIG. 9 illustrates an image of a pathological tissue after vascular dilatation with heat.

FIG. 10 illustrates a high-magnified image of the region X shown in FIG. 9.

### DESCRIPTION OF EMBODIMENTS

As follows is a detailed description of embodiments of a radiofrequency balloon catheter system proposed by the present invention with reference to the appended drawings.

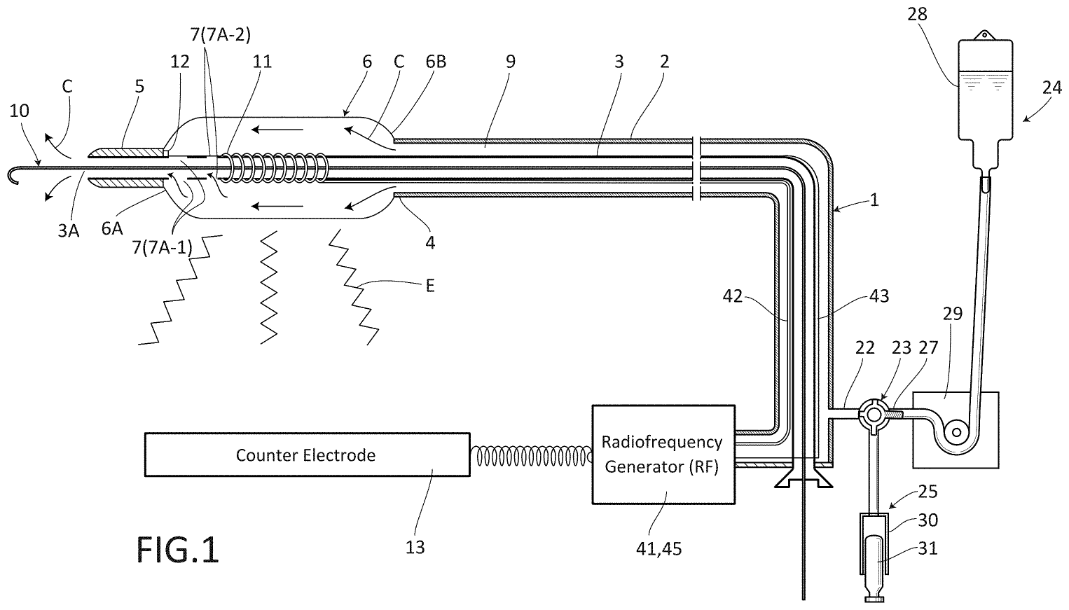

FIGS. 1 to 4 and 5A illustrate a main framework of the radiofrequency balloon catheter system according to an embodiment of the present invention. In the drawings, numerical symbol 1 denotes a cylindrical catheter shaft that can be inserted into a luminal organ. The catheter shaft 1 is composed of an outer tube shaft 2 and an inner tube shaft 3 which are hollow and slidable with each other in an anteroposterior direction. A deflatable and inflatable balloon 6 is provided between a vicinity of a distal portion 4 of the outer tube shaft 2 and a vicinity of a distal portion 5 of the inner tube shaft 3. The balloon 6 is formed into the shape of one with thin membrane which is made of a heat-resistant resin such as polyurethane, PET (polyethylene terephthalate) or the like, and has an appropriate elasticity. The balloon 6 is provided with necks 6A and 6B that are respectively arranged in the anterior and posterior portions of the balloon 6, each have a cylindrical shape and a thickness smaller than any other portions of the balloon, and are fixed to the catheter shaft 1. Further, inside the balloon 6, perfusion nozzles 7 used for perfusion are bored in the inner tube shaft 3.

Defined between the outer tube shaft 2 and the inner tube shaft 3 is a solution transport path 9 in communication with the inside of the balloon 6. When the balloon 6 is filled with a solution serving as a coolant C (normally, a cooled mixture of a non-ionic contrast agent and one of distillated water and dextrose in water) through the solution transport path 9, the balloon 6 is thus inflated into the shape of a rotatable body, e.g., substantially spherical shape, and at the same time, as the coolant C flows through a plurality of nozzles 7, it will then be discharged to the outside from discharge holes 3A that are opened at the distal end of the inner tube shaft 3. The numerical symbol 10 denotes a guide wire for guiding the balloon 6 to a target site, and the guide wire 10 is inserted through the inner tube shaft 3.

Arranged inside the balloon 6 are an electrode 11 for delivery of radiofrequency current and a temperature sensor 12. The electrode 11 for delivery of radiofrequency current is arranged as an electrode for radiating a radiofrequency electric field E, and is provided in such a coiled fashion that it is wound around the circumferential periphery of the inner tube shaft 3. Further, the electrode 11 for delivery of radiofrequency current has a monopolar structure, and is configured to deliver a radiofrequency current between itself and a counter electrode 13 provided outside the catheter shaft 1. When a current is applied thereto, then, there will be radiated the electric field E from the electrode 11 for delivery of radiofrequency current to the surroundings thereof.

A temperature sensor 12, serving as a temperature detection unit, is provided at a distal portion of the inner tube shaft 3 inside the balloon 6, and arranged anterior to the electrode 11 for delivery of radiofrequency current, the temperature sensor 12 being in contact with a balloon film in order to detect a temperature of a region in close proximity of the balloon surface. Further, though not shown in figure, there can be fixed not only the temperature sensor 12 but also electrodes for the monitoring, which are respectively provided on the anterior and posterior portions of the balloon 6, and are connected via electric wires to an impedance measurement device in order to measure the impedance across the balloon 6.

Outside the catheter shaft 1, a communication tube 22 is connected to a basal portion of the solution transport path 9 in a communicative manner. One port of a three-way cock 23 is coupled to the basal portion of this communication tube 22, and the remaining two ports of the three-way cock 23 are respectively coupled to a liquid transfusing unit 24 for inflating the balloon 6 and a syringe 25 for deflating the balloon 6. The three-way cock 23 has an operation piece 27 capable of being pivotally operated by the fingers such that one of the liquid transfusing unit 24 and the syringe 25 may come into a fluid communication with the communication tube 22, or eventually with the solution transport path 9 by the operation of the operation piece 27.

The liquid transfusing unit 24 is made up of a transfusion bottle 28 for reserving the coolant C and a liquid transfusing pump 29 in communication with the transfusion bottle 28. When the liquid transfusing pump 29 is activated with the liquid transfusing unit 24 and the communication tube 22 communicated with each other through the three-way cock 23, the coolant C, having reached from the transfusion bottle 28, is pumped out through the liquid transfusing pump 29 into the solution transport path 9, thereby turning the pressure at the inside of the balloon 6 to positive. A syringe 25, serving as a liquid recovering unit, includes a cylindrical body 30 connected to the three-way cock 23, and a movable piston 31 provided within the cylindrical body 30. If the piston 31 is pulled back with the syringe 25 being communicated with the communication tube 22 through the three-way cock 23, the solution is recovered from the inside of the balloon 6 via the solution transport path 9 into the inside of the cylindrical body 30, thereby turning the pressure inside the balloon 6 to negative.

Further, a radiofrequency generator 41 is provided outside of the catheter shaft 1. Within the balloon 6 are arranged the electrode 11 for delivery of radiofrequency current and the temperature sensor 12, which are electrically connected to the radiofrequency generator 41 respectively through the electric wires 42, 43 placed inside the catheter shaft 1. The radiofrequency generator 41 supplies radiofrequency energy, as an electric power, between the electrode 11 for delivery of radiofrequency current and the counter electrode 13 through the electric wire 42 to heat the whole of the balloon 6 filled with the solution. The radiofrequency generator 41 is provided with a thermometer (not shown) for measuring a temperature on the balloon 6 and displaying the same in response to a detection signal transmitted through the other electric wire 43 from the temperature sensor 12. Further, the radiofrequency generator 41 is configured to sequentially retrieve information on temperatures measured by the thermometer to determine a level of radiofrequency energy (output) to be supplied through the electric wire 42 to between the electrode 11 for delivery of radiofrequency current and the counter electrode 13. The electric wires 42, 43 are fixed along the inner tube shaft 3 over the entire axial length of the inner tube shaft 3.

According to the present embodiment, whilst the electrode 11 for delivery of radiofrequency current is used as a heating means for heating the inside of the balloon 6, it is not to be limited to any specific ones as long as it is capable of heating the inside of the balloon 6. For example, as a substitute for the electrode 11 for delivery of radiofrequency current and the radiofrequency generator 41, there may be employed any one of couples of an ultrasonic heating element and an ultrasonic generator, a laser heating element with a laser generator; a diode heating element with a diode power supply, and a nichrome wire heating element with a nichrome wire power supply unit.

Further, the catheter shaft 1 and the balloon 6 are all made of such a heat-resistant resin that can withstand heating without having any thermal deformation and the like when heating the inside of the balloon 6. The balloon 6 may take not only a spherical shape whose long and short axes are equal, but also any other shapes of any rotational bodies such as an oblate spherical shape whose short axis is defined as a rotation axis, a prolate spheroid whose long axis is defined as a rotation axis, or a bale shape. In any of these shapes, the balloon is made up of such an elastic member having a compliance that deforms when it comes in close contact with an inside wall of a luminal organ.

When the balloon is subject to a positive pressure as described above, the amount of the coolant C to be discharged through nozzles 7 to the outside of the balloon 6, that is, discharge rate of the solution from the inside of the balloon 6 can be adjusted by the output of the liquid transfusing pump 29. Preferably, a temperature control means 45 is provided for automatically and variably controlling radiofrequency energy generated from the electrode for delivery of radiofrequency current 11 and an output from the liquid transfusing pump 29, in response to a detection signal from the temperature sensor 12 such that the balloon 6 maintains a surface temperature of 45° C. or lower.

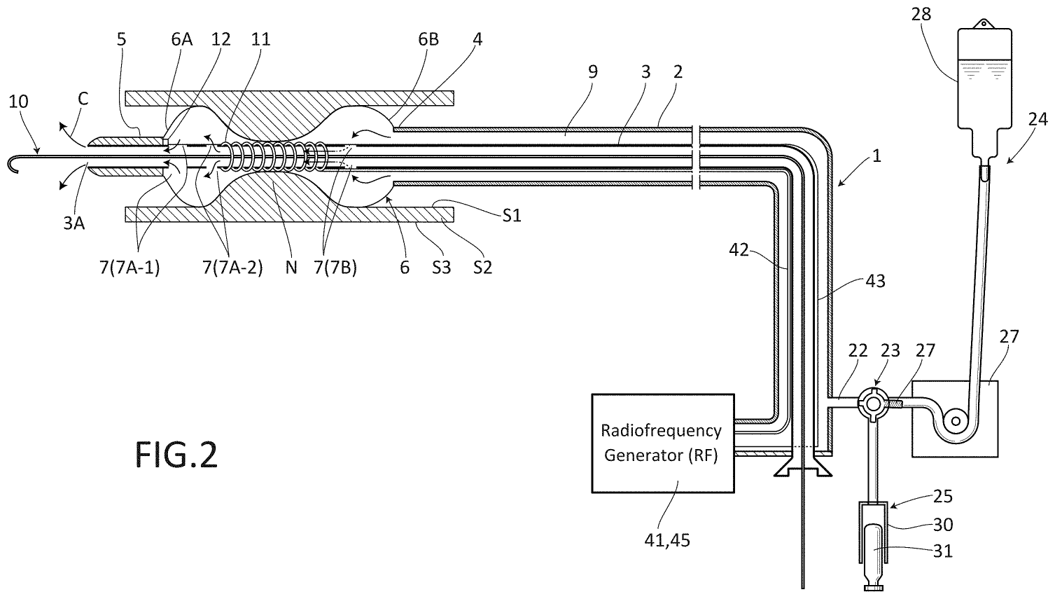

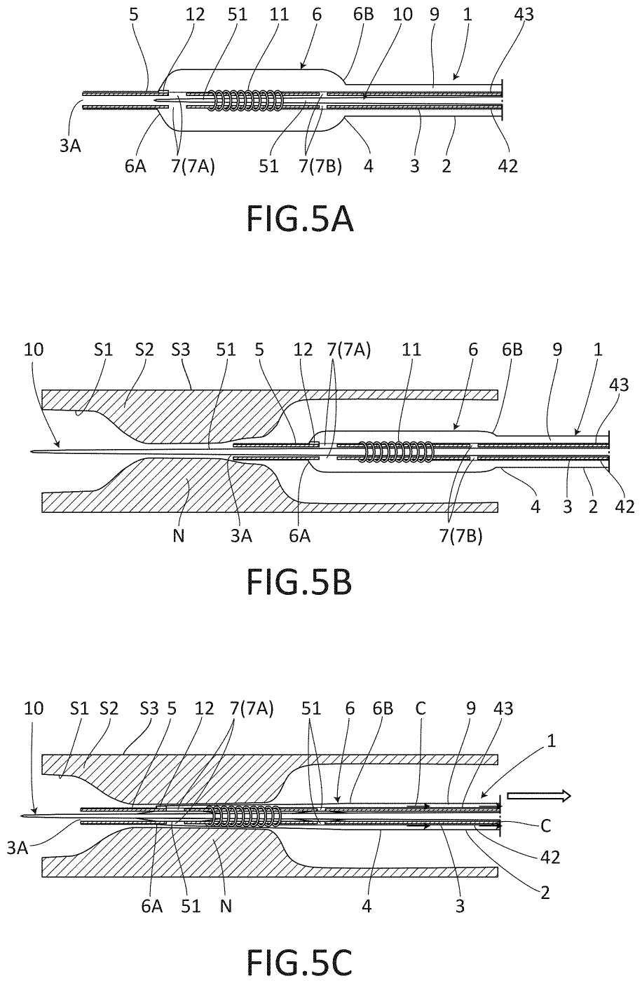

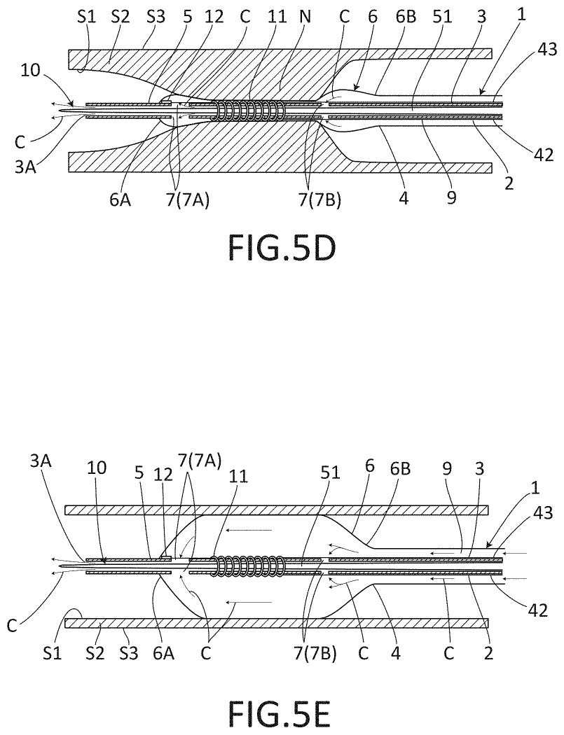

The nozzles 7 at least include distal nozzles 7A provided within the balloon at the distal portion of the inner tube 3 and arranged anterior to the electrode for delivery of radiofrequency current 12. Not only that, the nozzles 7 may also include proximal nozzles 7B provided at the proximal portion of the inner tube 3 and arranged posterior to the electrode for delivery of radiofrequency current 12. FIG. 1 illustrates an example in which the nozzles 7 are composed of a plurality of distal nozzles 7A-1 and 7A-2, which are respectively arranged in front and rear sides of the distal portion of the inner tube shaft 3, but the proximal nozzles 7B are not provided. In an example as illustrated in FIGS. 2 to 4, the nozzles 7 are composed of not only the above-mentioned distal nozzles 7A-1 and 7A-2 but also of a combination with the plurality of proximal nozzles 7B arranged at the proximal portion of the inner tube shaft 3. FIGS. 5A to 5E illustrate an example in which the nozzles 7 are composed of in combination with the distal nozzles 7A and the proximal nozzles 7B. Although not shown in any of the figures, the proximal nozzles 7B may be arranged in the front and rear sides of the proximal portion of the inner tube shaft 3 (not shown).

The guide wire 10, as illustrated in FIGS. 5A to 5E, is fully coated with a resilient elastic material 51 on the entire surface thereof, and is configured to be expanded or squeezed by an external force. Further, the guide wire 10 has a distal end portion having such a tapered shape that is gradually tapered toward the distal end. Owing to this configuration, as the distal end of the guide wire 10 slides forward to a position anterior to the discharge hole 3A of the inner tube shaft 3, the guide wire 10 comes into contact with and conforms with the lumen of the inner tube 3 provided with the nozzles 7 by the elastic expansion of the elastic material 51.

As to a method for implementing the above-discussed aspects, FIGS. 5B to 5E illustrate a description of the vascular dilation procedures using the radiofrequency balloon catheter system according to the present embodiment. In each of these figures and FIGS. 2 to 4, symbols S1, S2, and S3 respectively denote the intima, media and adventitia of a coronary artery. Symbol N denotes a vascular stenosis site.

Into the stenosis site N is intra-arterially inserted a guide sheath (not shown) through which the balloon catheter, including the catheter shaft 1 and the balloon 6, is further inserted into the coronary artery using the guide wire 10 (FIG. 5B). At the posterior end of the catheter shaft 1, the syringe 25 is connected to the three-way cock 23 connected to the outlet of the solution transport path 9 that is communicated with the inside of the balloon 6 so as to bring the syringe 25 and the solution transport path 9 in communication with each other. Under that condition, as the piston 31 is pulled back to strongly suction the inside of the balloon 6, a contacting portion between the guide wire and the nozzle 7 bored in the inner tube shaft 3 inside the balloon 6 is closed and then the balloon become subject to a negative pressure, thus causing the balloon to be strongly deflated. As a result, the balloon 6 is allowed to be inserted into the vascular stenosis site N (FIG. 5C).

Next, with the liquid transfusing pump 29 being connected to the communication tube 22 in communication with the solution transport path 9 such that the liquid transfusing pump 29 and the solution transport path 9 is brought into communication with each other through the three-way cock 23, the liquid transfusing pump 29 is activated to slowly inject the coolant C into the balloon 6 and, at the same time, the radiofrequency generator 41 is used to initiate a delivery of radiofrequency current between the counter electrode 13, placed on the surface of a body, and the electrode 11 for delivery of radiofrequency current that is provided within the balloon 6. The coolant C having reached to the inside of the balloon 6 passes through a hollow portion of the inner tube shaft 3 via the proximal nozzles 7B while dilating the proximal portion of the balloon 6, and then a portion of the coolant flows through the distal nozzles 7A to dilate the distal portion of the balloon, while the remaining portion flows straight through the hollow portion of the inner shaft 3 to be discharged via the discharge hole 3A at the end thereof to the outside (FIG. 5D). The coolant perfusing through the inner tube shaft 3 cools the electrode for delivery of radiofrequency current 11, which in turn prevents damage to intima S1 of a vessel, and irradiation of radiofrequency electric field, together with the enhancement of internal pressure inside the balloon 6, thermally dilates stenosis site N.

Here, when the liquid transfusing pump 29 raises the infusion rate of the coolant C, the balloon 6 becomes further inflated such that a squeezed portion, formed between the proximal portion and the distal portion of the balloon 6, becomes less squeezed. As the result, the coolant C inside the balloon passes mainly through a region within the balloon, and subsequently through the distal nozzles 7A into the hollow portion of the inner tube shaft 3, and then the coolant C is discharged through the distally arranged discharge hole 3A out to the outside. If the vascular stenosis site N, being in contact with the outer surface of the balloon 6, is not sufficiently dilated, then, the infusion rate of the coolant C is further increased to elevate the internal pressure of the balloon 6, or otherwise, radiofrequency output of the radiofrequency generator 41 is powered up to enhance the intensity of the electric field between the counter electrode 13 and the electrode 11 for delivery of radiofrequency current.

In this way, as illustrated in FIG. 5E, if the vascular stenosis site N becomes sufficiently dilated, the radiofrequency generator 41 stops delivering the radiofrequency current, and the guide wire 10 is inserted again to suction the coolant C, serving as an in-balloon fluid of the balloon 6, from the solution transport path 9 using the syringe 25 to deflate the balloon 6 to remove itself from the stenosis site N. After that, there will be performed a contrast study by way of the tip end of the catheter.

The radiofrequency balloon catheter system according to the present embodiment may be used not only for the treatment of vascular stenosis as explained above but also for stenosis of urethra, ureter, bile passage, or pancreas duct.

As illustrated in FIG. 2, according to the radiofrequency balloon catheter system provided with a plurality of distal nozzles 7A-1 and 7A-2 that are respectively arranged in the front and rear sides of the distal portion of the inner tube shaft 3, after having inserted the deflated balloon 6 into the vascular stenosis site N, in accordance with the procedure as explained above, the liquid transfusing pump 29 is first activated to infuse the coolant C into the balloon, and then the coolant C flows via the proximal nozzles 7B through the hollow portion of the inner shaft 3. The coolant then flows through the distal nozzles 7A-2 arranged in the rear side of the distal portion of the inner tube shaft 3 to thereby dilate the distal portion of the balloon 6, from which the coolant C passes into the nozzles 7A-1, arranged in the front side thereof, through the hollow portion of the inner shaft 3, and then passes via the distally arranged discharge hole 3A out to the outside.

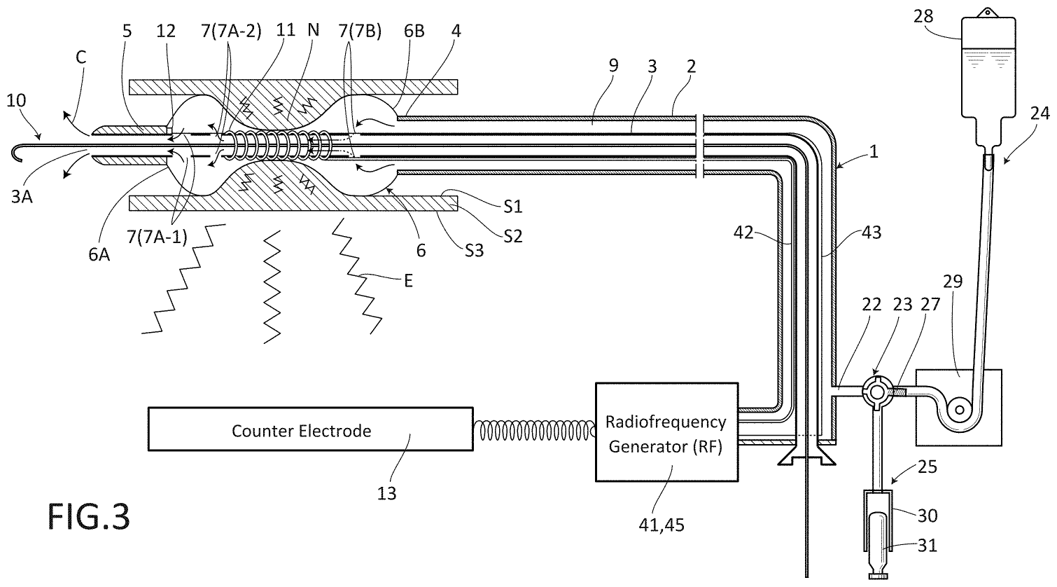

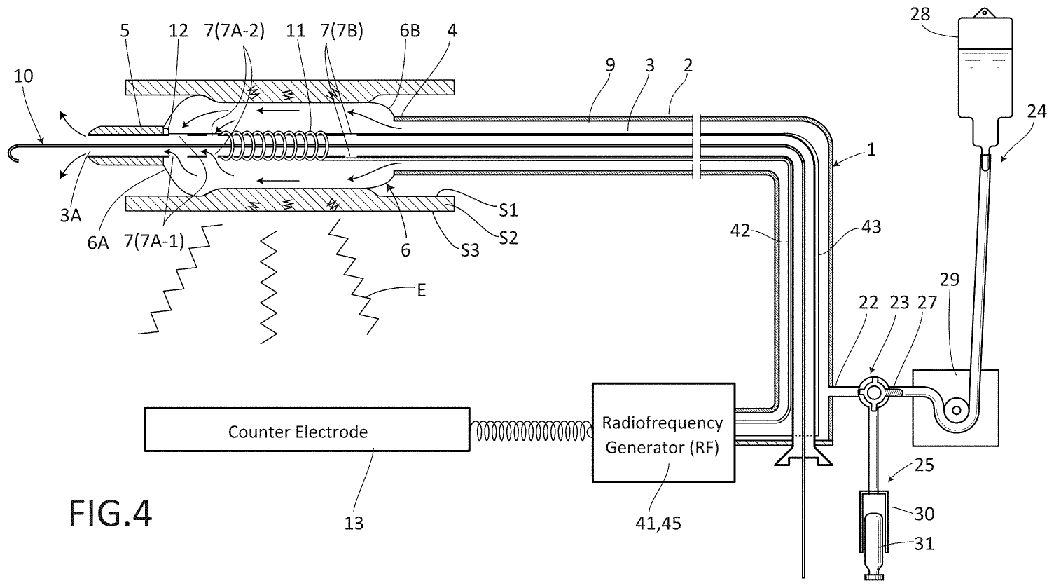

In this case, as shown in FIG. 3, when the radiofrequency generator 41 is connected to the counter electrode 13, and the radiofrequency generator 41 initiates delivery of radiofrequency current between the counter electrode 13 and the electrode 11 for delivery of radiofrequency current, irradiation of radiofrequency electric field, together with the enhancement of internal pressure inside the balloon 6, thermally dilate stenosis site N, while the coolant C, serving as a perfusate, flows through the hollow portion of the inner shaft 3 along the region between the proximal nozzles 7B and distal nozzles 7A-2, arranged in the rear side of the distal portion, to thereby cool the inside of the balloon 6 and the electrode for delivery of radiofrequency current 11. As the result, the intima S1 remains undamaged. Moreover, the media S2 is softened primarily for the collagen with heat at 45 to 50° C. to readily allow a stenosis site N in a blood vessel to be dilated even under a relatively low internal pressure of 3 to 6 atmospheres inside the balloon 6. As the balloon 6 dilates the stenosis site N, and the squeezed portion, formed between the proximal and distal portions of the balloon 6, becomes less squeezed, a large portion of the coolant C inside the balloon passes through a region within the balloon 6, and via the distal nozzles 7A-1, arranged in the front side of the distal portion, into the hollow portion of the inner tube 3, and then the coolant C is discharged through the distally arranged discharge hole 3A out to the outside (FIG. 4).

Note that the radiofrequency balloon catheter system as illustrated in FIGS. 2 to 4 may employ the guide wire 10 coated with the elastic material 51 as illustrated in FIGS. 5A to 5E. In any case, as the coolant C, serving as an in-balloon solution of the balloon 6, is suctioned through the solution transport path 9 with the distal end of the guide wire 10, having tapered shape, being slided to a position anterior to the discharge hole 3A of the inner tube shaft 3, the suctioning pressure makes the balloon 6 subject to a negative internal pressure to thereby elastically deform the elastic material 51 of the guide wire 10 and close the nozzles 7, allowing the balloon 6 to be strongly deflated.

The radiofrequency heating balloon catheter system of the present embodiment is provided with the electrode for delivery of radiofrequency current 11 inside the film-made balloon 6 arranged at a distal end of the catheter shaft 1, and the plurality of nozzles 7 for discharging in-balloon solution are bored in the inner tube shaft 3 inside the balloon 6. Hence, the electrode for delivery of radiofrequency current 11, arranged on the inner tube shaft 3 inside the balloon 6, is forcibly cooled by the perfusion flowing through the outside of the inner tube shaft 3, as well as through the inside of the inner tube shaft 3. Further, since the temperature sensor 12 is arranged in contact with an inner surface of the balloon 6 at a distal portion of the inner tube 3 inside the balloon 6, one is allowed to near accurately know the surface temperature of the balloon 6 through the radiofrequency generator 41 in response to a detection signal from the temperature sensor 12. That is, one is allowed to observe the surface temperature of the balloon 6 through the thermometer arranged in the radiofrequency generator 41 in a real-time manner.

Accordingly, in a case where the radiofrequency balloon catheter system is utilized for vascular application, radiofrequency energy to be supplied between the counter electrode 13 and the electrode for delivery of radiofrequency current 1 may be controlled to maintain the surface temperature of the balloon 6 at 45° C. or lower to thereby heat a luminal organ media at a temperature of 45 to 50° C. for 30 to 60 seconds without damaging endothelial cells in contact with the balloon 6. As the result, the collagen may be softened with minimal damages and stretched under a relatively low pressure of 3 to 6 atmospheres inside the balloon 6 which is pressurized by the liquid transfusing pump 29, thereby allowing the stenosis site to be dilated without having any vascular dissection.

Moreover, even after cessation of delivering a radiofrequency current through the radiofrequency generator 41 to the electrode for delivery of radiofrequency current 11, the liquid transfusing pump 29 continues to provide pressures and perfusion cooling inside the balloon 6 to thereby restrain the inflammatory response at the heat-treated site and to memorize the shape of collagen in a stretched state, thus allowing luminal organ to keep the dilated configuration.

The above-described sequence of steps can be achieved by manually controlling radiofrequency energy generated from the electrode for delivery of radiofrequency current 11 or the amount of supply of the coolant C to be pumped out from the liquid transfusing pump 29 into the balloon 6 while monitoring the thermometer arranged in the radiofrequency generator 41. Particularly, according to the present embodiment, the system includes a temperature control means 45 for variably and automatically controlling radiofrequency energy generated from the electrode for delivery of radiofrequency current 11 and the amount of supply of the coolant C to be pumped out from the liquid transfusing pump 29 into the balloon 6 such that the balloon 6 maintains a surface temperature, to be detected by the temperature sensor 12, at 45° C. or lower, which allows one to observe surface temperature of the balloon 6 in a real-time manner, thus eliminating the need of such manual control. The temperature control means 45 preferably is configured to provide the electrode for delivery of radiofrequency current 11 with energization time of 30 to 60 seconds.

According to the system for cooling the inside of the balloon 6 while heating the outside of the balloon 6, since the system employs a conventional balloon catheter members, as well as an electrode of fine wire, the system has an advantage of enabling a further application to a treatment of a narrow vascular stenosis site such as those for coronary angioplasty without significantly changing the profile of the balloon 6.

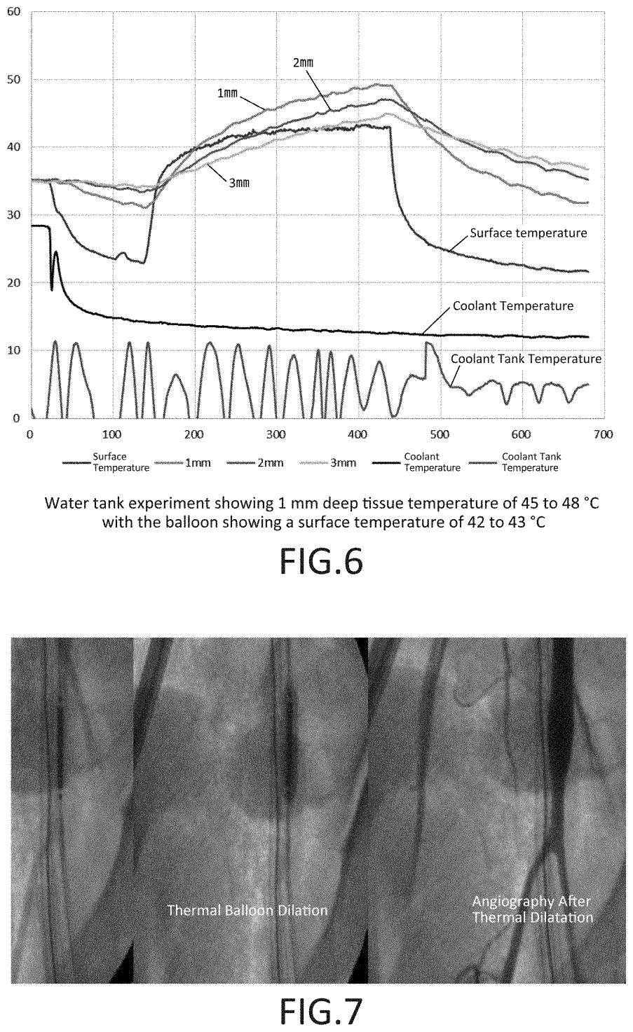

FIG. 7 explains one of various experiments on animals, illustrating a perspective image of the balloon catheter 1 interposed within a porcine iliac artery at a moment of being inflating and perfusing the inside of the balloon 6 with the coolant C, serving as a perfusate, being infused at the rate of 30 cc per minute under 3 to 6 atmospheres while delivering radiofrequency current at the output of 40 W for one minute. Angiography, performed after vascular dilatation by heat and pressure with the balloon, shows a blood vessel that has been dilated without any dissociation.

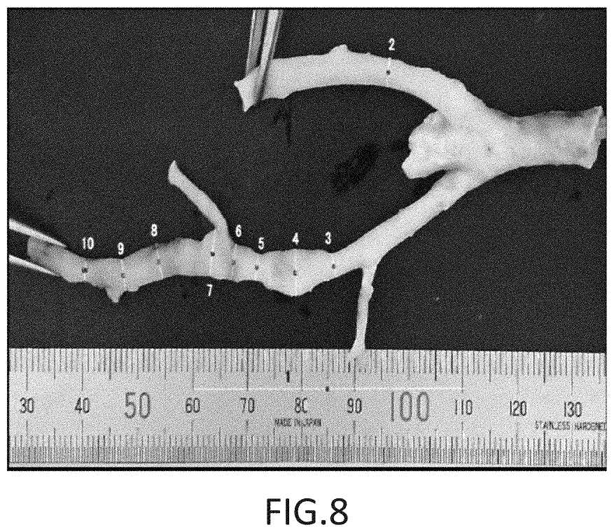

FIG. 8 illustrates an overall image of the tissue after angioplasty, showing an iliac artery having dilated from 3.2 mm to 6.1 mm in diameter.

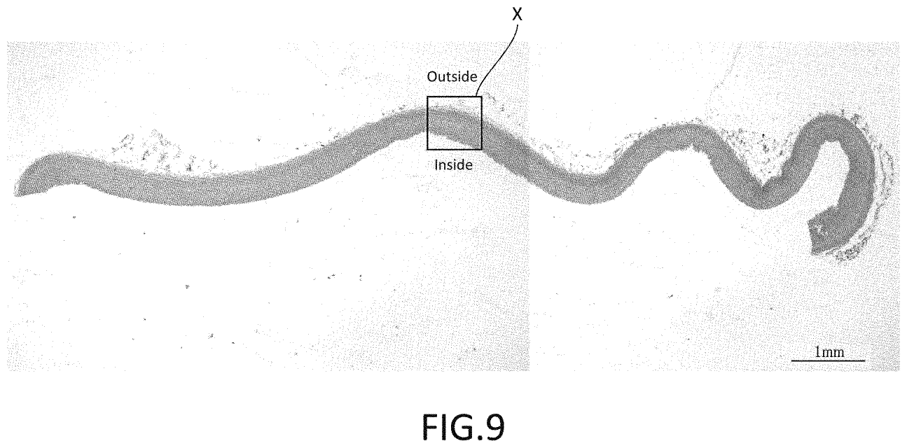

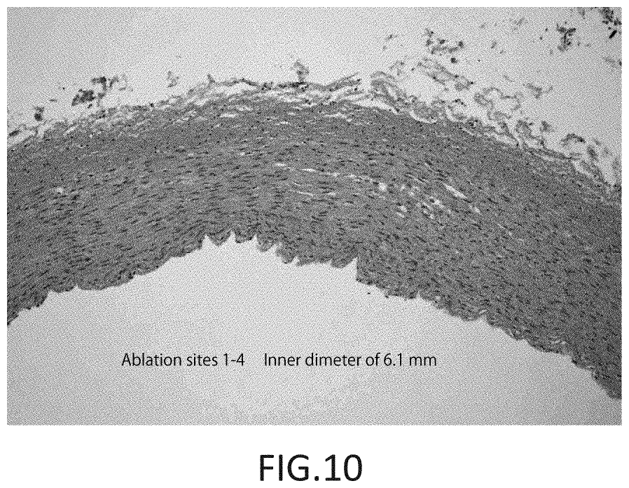

FIG. 9 illustrates an image of the pathological tissue after vascular dilatation with heat, showing degeneration focusing around the media cell. Nevertheless, the image shows a vascular wall having stretched without any vascular dissection, media dissection or intimal dissection. Also, FIG. 10 illustrates a magnified image of the region X in FIG. 9.

In this way, the radiofrequency balloon catheter system, according to the present embodiment, includes:

the catheter shaft 1 comprising the inner tube shaft 3, as the inner tube, and the outer tube shaft 2, as the outer tube;

the balloon 6 that is inflatable and deflatable and provided between the distal end portion 5 of the inner tube shaft 3 and the distal end portion 4 of the outer tube shaft 2;

the electrode for delivery of radiofrequency current 11 mounted on the inner tube shaft 3 and provided within the balloon 6;

the plurality of in-balloon perfusion nozzles 7 bored in the inner tube shaft 3 inside the balloon 6;

the temperature sensor 12 arranged at a distal portion of the inner tube shaft 3 inside the balloon 6 in contact with the film of the balloon 6;

the radiofrequency generator 41 provided with the thermometer 12 is connected to the electrode for delivery of radiofrequency current 11 and the temperature sensor 12 respectively via electric wires 42 and 43 within the catheter shaft 1;

the solution transport path 9 defined by the outer tube shaft 2 and the inner tube shaft 3 in communication with an inside of the balloon 6, and connected to the transfusing pump 29, serving as a perfusion pump, for feeding the coolant C into the balloon 6; and

the guide wire 10 that is coated with an elastic material 51 and insertable into the hollow portion of the inner tube shaft 3.

In this case, as the guide wire 10 is inserted into the inner tube shaft 3 of the catheter shaft 1, and the solution within the balloon 6 is suctioned through the solution transport path 9, the elastic material 51 of the guide wire 10 become deformed and extended to close the nozzles 7 bored in the inner tube shaft 3, allowing the balloon 6 to be deflated. Meanwhile, as the balloon 6 is injected with the coolant C serving as a perfusate, the balloon 6 become inflated to deform and squeeze the elastic material 51 of the guide wire 10 to thereby release the nozzles 7, thus discharging the perfusate via the nozzles 7 through the hollow portion of the inner tube shaft 3 to the outside. Upon delivery of radiofrequency current from the radiofrequency generator 41 to the electrode for delivery of radiofrequency current 11, the electrode for delivery of radiofrequency current 11 inside the balloon 6 uniformly radiates radio frequency electric field to thereby heat the surrounding of the balloon 6. In the meantime, the temperature sensor 12, arranged at a distal portion of the inner tube shaft 3 inside the balloon 6 in contact with a film of the balloon 6, allows one to monitor a value close to the surface temperature of the balloon 6. Accordingly, the radiofrequency energy generated from the electrode for delivery of radiofrequency current 11 and the amount of supply of the coolant C to be pumped out from the liquid transfusing pump 29 into the balloon 6 may be appropriately controlled to provide a radiofrequency balloon catheter system capable of softening and stretching collagen—an adhesive material of luminal organs—by pressure with minimal damages to wall-constituting cells.

Further, as illustrated in FIGS. 5A to 5E, as the plurality of nozzles 7, the distal nozzles 7A and the proximal nozzles 7B are respectively arranged at distal and proximal portions of the inner tube shaft 3 inside the balloon 6, where the distal nozzles 7A are formed greater in size than the proximal nozzles 7B, and perfusing greater amount of perfusate than the proximal nozzles 7B.

In this case, the inner tube shaft 3 inside the balloon 6 has the plurality of nozzles 7 bored through in a region extending from the distal portion of the inner tube shaft 3 to the proximal portion thereof, where the proximal nozzles 7B are formed smaller in size the distal nozzles 7A. Accordingly, even if the balloon 6 is tightly interposed in the stenosis site N, the coolant C, serving as a perfusate, inside the balloon 6 continues to flow via the proximal nozzles 7B through the hollow portion of the inner tube shaft 3 to thereby cool the electrode for delivery of radiofrequency current 11 from within. If, on the other hand, the balloon 6 is inflated from the stenosis site N, the coolant C inside the balloon 6 passes mostly through a region within the balloon 6 before being discharged via the distal nozzles 7A through the hollow portion of the inner tube shaft 3 to the outside thereof, thus effectively cooling the electrode for delivery of radiofrequency current 11, and the inside of balloon 6.

Further, according the present embodiment, the system is configured to perfuse a coolant C inside the balloon 6 such that such that radiofrequency electric field irradiation from the electrode for delivery of radiofrequency current 11 causes a temperature of a media of a target luminal organ to be raised from 45 to 50° C. while regulating a temperature of an intima of a target tissue that is in contact with the balloon to 45° C. or lower. This feature can be achieved, preferably, by means of the temperature control means 45 which, in response to a surface temperature of the balloon 6 detected by the temperature sensor 12, automatically controls radiofrequency output from the electrode for delivery of radiofrequency current 11 and the supplying rate of the coolant C per unit time to be pumped out from the liquid transfusing pump 29 into the balloon 6 such that an intima of a target tissue, in contact with the balloon 6, maintains a temperature of 45° C. or lower and that a media of the target luminal organ is heated to a temperature of 45 to 50° C.

In this case, if the balloon 6 has a length of 20 mm and a diameter of 2.5 to 5 mm, then, the perfusion rate of the coolant C inside the balloon 6 is set at 10 to 50 cc per minute, and the radiofrequency output from the electrode for delivery of radiofrequency current 11 is set as 20 to 80 W. In such condition, the temperature of an intima of a target tissue, in contact with the film of the balloon 6, is maintained at 45° C. or lower, while a radiofrequency electric field irradiated thereon heats the media of the target luminal organ to the temperature of 45 to 50° C. As the result, the media collagen can be softened without having any major influence, such as coagulative necrosis, on the cell constituent.

Further, according to the present embodiment, the system is configured to maintain an intima of the target tissue, in contact with the balloon 6, at a temperature of 45° C. or lower and to allow the electrode for delivery of radiofrequency current 11 to deliver the current for a period of 30 to 60 seconds for minimizing damages to intima. This feature can be achieved preferably by means of the temperature control means 45 which, in response to a surface temperature of the balloon 6 detected by the temperature sensor 12, automatically controls radiofrequency output from the electrode for delivery of radiofrequency current 11 by setting the energization time for 30 to 60 seconds such that an intima of the target tissue, in contact with the balloon 6, is maintained at the temperature of 45° C. or lower.

As the result, the temperature of an intima of the target tissue, in contact with the balloon 6, is allowed to be kept at 45° C. or lower to thereby minimize damages to intima.

Further, according to the present embodiment, the system is configured to heat a media of a target luminal organ at a temperature of 45 to 50° C. for 30 to 60 seconds to soften the collagen fibers while minimizing damages to the media cells in order to dilate a stenosis site using the balloon 6 inflated under a relatively low pressure of 3 to 6 atmospheres without causing any dissociations or dissections. This feature can be achieved by means of the temperature control means 45 which, in response to a surface temperature of the balloon 6 detected by the temperature sensor 12, set the energization time of the electrode for delivery of radiofrequency current 11 for the period of 30 to 60 seconds, and automatically controls the radiofrequency output from the electrode for delivery of radiofrequency current 11, as well as the supplying rate of the coolant C per unit time to be pumped out from the liquid transfusing pump 29 into the balloon 6 such that a media of the target luminal organ is heated to a temperature of 45 to 50° C. and that the balloon 6 maintains internal pressure of 3 to 6 atmospheres.

In this case, the system is configured to control radiofrequency output from the electrode for delivery of radiofrequency current 11 for heating a media of luminal organ at a temperature of 45 to 50° C. for 30 to 60 seconds to soften collagen fibers while minimizing damages to media cells and to control the supplying rate of coolant C to be pumped out per unit time from the liquid transfusing pump 29 into the balloon 6 in order to allow a stenosis site to be dilated using the balloon 6 inflated at relatively low pressure of 3 to 6 atmospheres without causing any dissection in a target tissue media or vascular recoil. Accordingly, no complication of acute vascular occlusion occurs, and restenosis can be minimized even after the dilation.

According to the present radiofrequency balloon catheter system, wherein the radiofrequency generator 41 is preferably configured to intermittingly, but not continuously, deliver a radiofrequency current to the electrode for delivery of radiofrequency current 11 or to deliver the same inconstantly with variations in intensity. As the result, by delivering radiofrequency current intermittingly or with variations in intensity, a tissue in contact with the balloon 6 can be heated up to a high deep temperature.

Although not shown in any of these figures, there may be provided impedance measurement electrodes in front and back of the balloon 6 to measure an impedance between them by the impedance measurement device. In this case, the electrodes are installed in front and back of the balloon via electric wires to which the impedance measurement device is connected. This impedance measurement device allows one to monitor a change exerted on tissues by the radiofrequency heating.

The present invention shall not be limited to the embodiments described above, and various modified embodiments are possible within the scope of the present invention. The radiofrequency balloon catheter system of the present invention can be used for dilation of stenosis sites in hollow organs such as urethra, ureter, pancreas duct, trachea, esophagus. Further, the catheter shaft 1 and the balloon 6 may have other various shapes conforming to the sites to be treated, and shall not be limited to those described in the foregoing embodiments.

### DESCRIPTION OF THE REFERENCE NUMERAL

1 catheter shaft
2 outer tube shaft (outer tube)
3 inner tube shaft (inner tube)
6 balloon
7 nozzles
9 solution transport path
10 guide wire
11 electrode for delivery of radiofrequency current
12 temperature sensor
29 liquid transfusing pump (perfusion pump)
41 radiofrequency generator (thermometer)
42 electric wire
43 electric wire
51 elastic material

1. A radiofrequency balloon catheter system comprising:
a catheter shaft including an inner tube and an outer tube;
a balloon that is inflatable and deflatable and provided between distal ends of the inner tube and the outer tube;
an electrode for delivery of radiofrequency current that is provided within the balloon;
a plurality of in-balloon perfusion nozzles bored in the inner tube inside the balloon;
a temperature sensor arranged at a distal portion of the inner tube inside the balloon, said temperature sensor being in contact with a film of the balloon;
a radiofrequency generator and a thermometer respectively connected to the electrode for delivery of radiofrequency current and the temperature sensor via electric wires within said catheter shaft;
a solution transport path defined by the outer tube and the inner tube, said solution transport path being in communication with an inside of the balloon, and connected to a perfusion pump for feeding a coolant into the balloon; and
a guide wire that is coated with an elastic material and insertable into said inner tube.

2. The radiofrequency balloon catheter system according to claim 1, wherein the plurality of in-balloon perfusion nozzles are arranged at distal and proximal portions of the inner tube, said nozzles arranged at the distal portion being formed greater in size than the nozzles at the proximal portion.

3. The radiofrequency balloon catheter system according to claim 1, wherein the system is configured to perfuse a coolant inside the balloon such that such that radiofrequency electric field irradiation causes a temperature of a media of a target luminal organ to be raised from 45 to 50° C. while regulating a temperature of an intima of a target tissue that is in contact with the balloon to 45° C. or lower.

4. The radiofrequency balloon catheter system according to claim 1, wherein the system is configured to maintain an intima of a target tissue, in contact with the balloon, at a temperature of 45° C. or lower and to set an energization time for 30 to 60 seconds.

5. The radiofrequency balloon catheter system according to claim 1, wherein the system is configured to heat a media of a target luminal organ at a temperature of 45 to 50° C. for 30 to 60 seconds to soften collagen fibers while minimizing damages to the media cells in order to dilate a stenosis site using the balloon inflated under a low pressure of 3 to 6 atmospheres without causing any dissociations or dissections.

6. The radiofrequency balloon catheter system according to claim 1, wherein the system is configured to intermittingly deliver a radiofrequency current to the electrode for delivery of radiofrequency current or to deliver the same with a variation in intensity.

7. The radiofrequency balloon catheter system according to claim 1, wherein the electrode is installed in front and back of said balloon to measure an impedance therebetween.
